(19) United States
(12) Patent Application Publication
(10) Pub. No.: US 2024/0343785 A1
(43) Pub. Date: Oct. 17, 2024

(54) INTRACELLULAR ANTIBODY AND PREPARATION METHOD AND USE THEREOF

(71) Applicant: Jinan University, Guangzhou (CN)

(72) Inventors: Sen Yan, Guangzhou (CN); Shihua Li, Guangzhou (CN); Caijuan Li, Guangzhou (CN); Xiaojiang Li, Guangzhou (CN); Yingqi Lin, Guangzhou (CN)

(21) Appl. No.: 18/611,706

(22) Filed: Mar. 21, 2024

(30) Foreign Application Priority Data
Mar. 22, 2023 (CN) 202310281290.0

(51) Int. Cl. C07K 16/18; A61K 38/00; C07K 14/705

(52) U.S. Cl. CPC C07K 16/18; C07K 14/70596; A61K 38/00; C07K 2317/80; C07K 2319/02

(57) ABSTRACT

Disclosed are an intracellular antibody (intrabody) and a preparation method and a use thereof. The intrabody includes a fragment of the variable region of the heavy chain (VH) of a monoclonal antibody that recognizes mutant huntingtin (mHTT) and a signal sequence of lysosome-associated membrane protein I (LAMP1). The VH fragment includes an amino acid sequence as shown in any one of SEQ. ID NO. 1-3. The intrabody described in this application possesses a bidirectional recognition function, enabling it to bind to and recognize mHTTs as well as specifically recognize lysosomes, thereby realizing the targeted degradation of mutant proteins and improving the degradation efficiency of the mutant proteins.

### CROSS-REFERENCE TO RELATED APPLICATIONS

This application claims priority of Chinese Patent Application No. 202310281290.0, filed on Mar. 22, 2023, the entire contents of which are incorporated herein by reference.

### INCORPORATES BY REFERENCE OF SEQUENCE LISTING XML

A XML file is incorporates by reference. The file name is HBJS-US-1-08.xml; the creation date is Mar. 20, 2024, and the size of the file is 20311 bytes.

### TECHNICAL FIELD

This application relates to the field of antibody engineering and disease prevention and treatment, in particular to an intracellular antibody (intrabody) and a preparation method and a use thereof.

### BACKGROUND

Neurodegenerative diseases encompass a group of disorders characterized by the progressive loss of neuronal structure or function, leading to dysfunction of the nervous system and eventually neuronal death. These diseases deteriorate over time due to the gradual loss of neurons or their myelin sheaths, ultimately resulting in neurological impairment. Common examples of neurodegenerative diseases include Huntington's disease (HD), Alzheimer's disease (AD), Parkinson's disease (PD), and amyotrophic lateral sclerosis (ALS). While a few familial neurodegenerative diseases have well-defined causative genes, the underlying mechanisms for most diseases remain unclear, and effective treatment options are lacking. However, the accumulation of mutant proteins leads to the formation of aggregates, a common pathological feature observed during the development of these neurodegenerative diseases. The mutant proteins disrupt the cellular microenvironment and affect the transport of intracellular and extracellular substances in the central nervous system, ultimately causing damage and death of nerve cells. Consequently, the clearance of these mutant proteins plays a crucial role in the treatment of such diseases.

HD is a rare inherited neurodegenerative disease characterized by progressive movement disorders, psychiatric abnormalities, and cognitive impairments. The global prevalence of this disease is estimated to be 2.7 per 100,000 individuals. HD follows an autosomal dominant inheritance pattern and is caused by an abnormal amplification of the cytosine-adenine-guanine (CAG) trinucleotide repeat sequences in the huntingtin (HTT) gene, located at 4p16.3. In individuals with the normal HTT gene, the CAG repeat sequences are typically less than 36. However, when the CAG repeats exceed 36, an expanded polyglutamine (polyQ) is formed in the HTT protein, leading to the generation of misfolded mutant HTTs (mHTTs). These mHTTs accumulate in the nuclei and cytoplasm of neurons, abnormally interact with other proteins or molecules, and form insoluble aggregates or inclusions in the brains of HD patients, thereby contributing to the development of the disease.

Despite significant progress in understanding the pathogenesis of HD, effective therapeutic methods for this disease are still lacking. Extensive research on polyQ diseases has revealed substantial damage to various cellular functions caused by abnormally expanded polyQ, leading to a prevailing theory that inhibiting the expression of these abnormal proteins could be a promising approach for curing HD. Consequently, scientists have devoted considerable efforts to developing therapeutic strategies that can reduce or prevent the expression of mHTTs in animal models of HD. One such approach involves RNA interference (RNAi)-based therapies, including small interfering RNA (siRNA), short hairpin RNA (shRNA), and microRNA (miRNA), which are complexed with vectors or delivered via viral vectors to the central nervous system. Among these approaches, antisense oligonucleotides (ASOs) have shown the most potential by effectively inhibiting the expression of mHTTs. Several studies have reported therapeutic effects of ASOs in different disease models, and excitingly, clinical trials have demonstrated that intrathecal administration of ASOs can reduce the levels of mHTTs in the cerebrospinal fluid of HD patients. While these treatment methods have demonstrated varying degrees of success in targeting mRNA levels and inhibiting transcription and translation of mHTTs, they do not directly target the mHTT proteins, which are the primary toxic products contributing to neurodegeneration. Consequently, there is a pressing need to identify a therapeutic approach that can selectively and effectively remove these mHTT proteins.

Currently, several studies have reported the use of intrabodies targeting different regions of the HTT protein. However, these studies have revealed certain limitations: (1) Intrabodies designed to target the Exon1-polyQ region have shown effectiveness at the cellular-level but were found to promote aggregate formation and accelerate cell death. (2) Intrabodies targeting the Exon1-polyP region have demonstrated recognition and varying reduction of mHTT levels in cells and mice. However, due to the presence of polyP regions in many genes, non-specific binding may occur, potentially affecting the function of other normal proteins. (3) Intrabodies targeting the Exon1 N-terminal region have shown low efficiency in targeting soluble mHTT proteins. (4) Intrabodies targeting mHTT aggregates have been verified to reduce the aggregates, but their recognition efficiency significantly decreases when the conformation of mHTT changes. Additionally, these intrabody fragments are approximately 30 kDa in size, making them challenging to manipulate, and the efficiency of mHTT degradation by these intrabodies through the cell's self-degradation system is low.

Currently, there is an urgent need to discover a medication that can selectively bind to and efficiently clear mutant huntingtin (mHTT) proteins and their aggregates.

### SUMMARY

Based on this, it is necessary to provide an intrabody specifically recognizing and binding to mHTT proteins and aggregates thereof, and a preparation method for the intrabody and use thereof.

The specific technical solutions are as follows.

An intrabody includes a fragment of a variable region of heavy chain (VH) of a monoclonal antibody recognizing an mHTT and a signal sequence of a lysosome-associated membrane protein I (LAMP1), the fragment of the VH including an amino acid sequence shown in any one of SEQ. ID NO. 1-3.

In some of embodiments, the fragment of the VH includes the amino acid sequence shown in SEQ. ID NO.3.

In some of embodiments, the signal sequence of the LAMP1 includes an amino acid sequence shown in SEQ. ID NO.4.

A preparation method for an intrabody includes the steps of:
constructing a recombinant expression vector plasmid containing a nucleotide sequence shown in any one of SEQ. ID NO.5-7 and containing a nucleotide sequence shown in SEQ. ID NO.8; and
transforming the recombinant expression vector plasmid into a recipient bacterium or recipient cell for culture and expression to obtain an intrabody.

In some of embodiments, a construction method for the recombinant expression vector plasmid includes the steps of:
synthesizing a target fragment, the target fragment containing a nucleotide sequence shown in any one of SEQ. ID NO.5-7 and containing a nucleotide sequence shown in SEQ. ID NO.8;
taking the target fragment as a template, taking sequences shown in any group of SEQ. ID NO. 9-10, SEQ. ID NO.18-19 and SEQ. ID NO. 20-21 as primers to amplify the target fragment, and adding EcoRI and AgeI restriction enzyme cutting sites; and
linking an amplified target fragment into an expression vector to obtain a recombinant expression vector plasmid.

In some of embodiments, the target fragment further contains a nucleotide sequence of an HA tag protein; and alternatively, the target fragment contains at least two nucleotide sequences including HA tag proteins, the nucleotide sequence of the HA tag protein being shown in SEQ. ID NO.11.

In some of embodiments, the nucleotide sequence of the target fragment is a sequence shown in any one of SEQ. ID NO. 12-14.

A gene encodes the expression of the above intrabody.

In some of embodiments, the nucleotide sequence shown in any one of SEQ. ID NO.5-7, and the nucleotide sequence shown in SEQ. ID NO.8 are contained.

A recombinant expression vector contains the gene described in any one of the above.

A use of an intrabody described in any one of the above, an intrabody obtained by a preparation method described in any one of the above, a gene described above or a recombinant expression vector described above is in any one of the following:
(1) in the preparation of a product for specifically recognizing and/or specifically binding to mutant HTT proteins and aggregates thereof;
(2) in the preparation of a product for degrading the mutant HTT proteins and aggregates thereof;
(3) in the preparation of a product for restoring lysosomal function; and
(4) in the preparation of a medicament for the treatment of a neurodegenerative disease.

A small molecule medicament includes an intrabody according to any one of the above, an intrabody obtained by a preparation method according to any one of the above, a gene according to any one of the above, or a recombinant expression vector described above.

Compared with the prior art, the present disclosure has the following beneficial effects.

1. This application successfully constructs a smaller Intrabody that can specifically recognize and bind to soluble mHTT and insoluble mHTT aggregates, enabling the possibility of making them into a small molecule peptide medicament for clinical conversion.

2. The Intrabody has a bidirectional recognition function, which can not only recognize and bind to mHTTs, but also specifically recognize lysosomes, thereby realizing the targeted degradation of mutant proteins and improving the degradation efficiency of mutant proteins.

3. The Intrabody can effectively restore lysosomal function, restore lysosomal enzyme activity in diseased mice, and ensure normal lysosomal degradation.

4. The Intrabody can well clear the expressed mHTT proteins and the formed aggregates, which provides a potential method for the treatment of HD at the middle and later stage and also provides a new idea and therapeutic method for the treatment of other neurodegenerative diseases.

### BRIEF DESCRIPTION OF THE DRAWINGS

FIG. 1 shows a schematic diagram of an scFv-mEM48-SM3 plasmid.

FIG. 2 shows the mEM48 immunofluorescence analysis of 293T cells transfected with three kinds of Intrabodies, determining that scFv-mEM48-SM3 can reduce aggregates produced by mHTTs more effectively.

FIG. 3 shows the Western blot (WB) analysis of 293T cells transfected with three kinds of Intrabodies, determining that scFv-mEM48-SM3 can reduce mHTTs more effectively.

FIG. 4A shows whether mHTTs of 293T cells transfected with intrabodies by the WB analysis enter lysosomes for degradation, and specifically shows a WB diagram for verifying whether the Intrabody carries mHTTs into lysosomes for degradation, where samples in WTpro group, 150Q+HA pro group and 150Q+SM3 pro group are the extracted total proteins, and the results show that the amount of mHTTs in the total proteins of cells transfected with Intrabodies is significantly reduced, indicating that Intrabody can effectively reduce mHTTs; samples in WT lys group, 150Q+HA lys group and 150Q+SM3 lys group are the extracted lysosomes, and the results show that mHTTs in lysosomes of cells transfected with Intrabodies are significantly increased, indicating that Intrabody carries mHTTs into lysosomes for degradation; and LAMP1, LAMP2, P62 and LC3A/B are lysosome-associated Makers, for identifying the quality of lysosome extraction, indicating that the quality of lysosome extraction has no problem; and

FIG. 4B is a quantitative diagram of FIG. 4A.

FIG. 5A shows the reduction of mHTTs after the WB analysis of the 120Q-293T stable cell line transfected with Intrabody, and specifically shows the detection results of 1C2 antibody, the 1C2 antibody recognizing mHTTs and specifically recognizing polyQ; and

FIG. 5B shows the reduction of mHTTs after the WB analysis of the 120Q-293T stable cell line transfected with Intrabody, and specifically shows the detection results of Mem48 antibody.

FIG. 6 shows the activation of lysosomes after the WB analysis of the 120Q-293T stable cell line transfected with Intrabody.

FIG. 7 shows the expression of Intrabody in the striatum of HD-KI-140Q mice after brain injection is detected by the expression of HA.

FIG. 8 shows the reduction of aggregates at the striatum in HD-KI-140Q mice after the mEM48 immunofluorescence analysis of the brain injection of Intrabody.

FIG. 9A shows the reduction of mHTT proteins and aggregates thereof at the striatum in HD-KI-140Q mice after the WB analysis of the brain injection of Intrabody, and specifically shows the detection results of 1C2 antibody;

FIG. 9B shows the reduction of mHTT proteins and aggregates thereof at the striatum in HD-KI-140Q mice after the WB analysis of the brain injection of Intrabody, and specifically shows the detection results of Mem48 antibody; and

FIG. 9C is a quantitative diagram of FIGS. 9A and 9B.

FIG. 10A shows the activation of lysosomes in HD-KI-140Q mice after the WB analysis of the brain injection of Intrabody, and specifically shows a WB diagram; and

FIG. 10B is a quantitative diagram of FIG. 10A, LAMP1, P62, LC3 being lysosome-associated Makers.

FIG. 11 shows the expression of Intrabody in the striatum of HD-KI-140Q mice after orbital intravenous injection is detected by the expression of HA.

FIG. 12 shows the reduction of aggregates at the striatum in HD-KI-140Q mice after the mEM48 immunofluorescence analysis of the orbital intravenous injection of Intrabody.

FIG. 13A shows the reduction of mHTT proteins and aggregates thereof in the brain of HD-KI-140Q mice after the WB analysis of the orbital intravenous injection of Intrabody, and specifically shows the detection results of 1C2 antibody;

FIG. 13B shows the reduction of mHTT proteins and aggregates thereof in the brain of HD-KI-140Q mice after the WB analysis of the orbital intravenous injection of Intrabody, and specifically shows the detection results of Mem48 antibody; and

FIG. 13C is a quantitative diagram of FIGS. 13A and 13B.

FIG. 14 shows whether the mHTT proteins in the brains of HD-KI-140Q mice enter lysosomes for degradation after the WB analysis of the orbital intravenous injection of Intrabody.

FIG. 15 shows whether the total acid enzyme activity of lysosomes is affected in the brains of HD-KI-140Q mice after the enzyme-linked immunosorbent assay (ELISA) analysis of the brain injection of Intrabody.

FIG. 16 shows whether the total acid enzyme activity of lysosomes is affected in the brains of HD-KI-140Q mice after the ELISA analysis of intravenous injection of Intrabody.

### DETAILED DESCRIPTION

To facilitate the understanding of this application, this application will be described more comprehensively below. However, this application can be implemented in many different forms and is not limited to the embodiments described herein. On the contrary, these embodiments are provided to make the disclosure of this application more thorough and comprehensive.

Unless otherwise defined, all technical and scientific terms used herein have the same meaning as commonly understood by those skilled in the technical field of this application. The terms used herein in the specification of this application are only for the purpose of describing specific embodiments, and are not intended to limit this application.

### Term Interpretation

A term “and/or” refers to including any and all combinations of one or more of the associated listed items.

A term “mHTT” refers to a mutated HTT protein.

A term “Lys” refers to a lysosome.

The term “intrabody” refers to a new class of engineered antibody that is expressed in non-lymphocytes and can be localized in subcellular compartments (e.g. nucleus, cytoplasm or certain organelles), specifically interfering with or blocking the activity, processing or secretion processes of a target molecule, thereby exerting its biological functions. In other words, it is a small antibody fragment directed against an intracellular antigen. It is an immunoglobulin that targets intracellular antigens, has high affinity, strong specificity of binding, and can be stably expressed in specific subcellular organs. Intrabody mainly exists in two forms: scFv and Fab. Because of its simple molecular structure, the scFv antibody maintains its affinity for antigen and is convenient for recombinant operation in vitro, which has become the most commonly used antibody form in intrabody technology. For different experimental purposes, scFv can be artificially localized and expressed in various subcellular compartments by modifying an N-terminal or C-terminal of scFv protein.

One embodiment of this application provides an intrabody, including a fragment of a VH of a monoclonal antibody recognizing an mHTT and a signal sequence of an LAMP1, the fragment of the VH including an amino acid sequence shown in any one of SEQ. ID NO.1-3.

In one specific example, the fragment of the VH includes the amino acid sequence shown in SEQ. ID NO.3.

In one specific example, an amino acid sequence of the signal sequence of the LAMP1 includes GYQTI, SEQ. ID NO.4.

An implementation of this application provides a preparation method for an intrabody. A signal sequence of LAMP1 is added after specifically recognizing an antibody fragment of mHTT, so that the Intrabody has a bidirectional function, i.e. specifically recognizing and binding to mHTT proteins and aggregates thereof, and carrying mHTT specificity after binding to mHTT into lysosomes for degradation, improving the mHTT degradation efficiency, and having a lysosomal repair function. Specifically, the following steps a to b are included.

At step a: a recombinant expression vector plasmid containing a nucleotide sequence shown in any one of SEQ. ID NO.5-7 and containing a nucleotide sequence shown in SEQ. ID NO.8 is constructed.

In one specific example, a construction method for the recombinant expression vector plasmid includes the following steps 1 to 3.

At step 1, a target fragment is synthesized, the target fragment containing the nucleotide sequence shown in any one of SEQ. ID NO.5-7 and containing the nucleotide sequence shown in SEQ. ID NO.8.

In one specific example, the target fragment further contains a nucleotide sequence of an HA tag protein. The HA tag protein is introduced to facilitate the subsequent detection for Intrabody.

Alternatively, at least two HA tag proteins are contained, and the nucleotide sequence of the HA tag protein is shown in SEQ. ID NO.11

In one specific example, the nucleotide sequence of the target fragment is a sequence shown in any one of SEQ. ID NO. 12-14.

At step 2, the target fragment is taken as a template, nucleotide sequences shown in any group of SEQ. ID NO. 9-10, SEQ. ID NO.18-19 and SEQ. ID NO. 20-21 are taken as primers to amplify the target fragment, and EcoR I and Age I restriction enzyme cutting sites are added. Those skilled in the art perform amplification according to conventional polymerase chain reaction (PCR) techniques.

The primer shown in SEQ. ID NO. 9-10 can be used to amplify a target fragment scFv-mEM48-SM3; the primer shown in SEQ. ID NO.18-19 can be used to amplify a target fragment scFv-mEM48-SM1; and the primer shown in SEQ. ID NO. 20-21 can be used to amplify a target fragment scFv-mEM48-SM2.

At step 3, an amplified target fragment is linked into an expression vector to obtain a recombinant expression vector plasmid.

In one specific example, the expression vector may be an adeno-associated virus (AAV), such as an ssAAV.CMV.EGFP.WPRE.SV40pA vector.

At step b: the recombinant expression vector plasmid is transformed into a recipient bacterium or recipient cell for culture and expression to obtain an intrabody.

In one specific example, the recipient bacterium may be selected from Escherichia coli, such as a competent cell of Escherichia coli. 293T cells can be selected as the recipient cells.

One implementation of this application also provides a gene encoding the expression of the intrabody. Any gene that can encode and express the intrabody described above belongs to the content of this application. Alternatively, the gene contains the nucleotide sequence shown in any one of SEQ. ID NO.5-7, and the nucleotide sequence shown in SEQ. ID NO.8.

Those skilled in the art are to know that in the gene for expressing the intrabody provided in this application, in addition to the above gene fragments, a nucleotide sequence such as a promoter, an enhancer and a non-coding region can also be included to realize the object to improve the performance of the gene in terms of expression amount, expression efficiency and product activity. In addition, the gene may also include a nucleotide sequence having a tag for facilitating detection of product expression.

One implementation of this application also provides a recombinant vector containing the gene described in any one of the above. Alternatively, a nucleotide sequence shown in any one of SEQ. ID NO.5-7, and a nucleotide sequence shown in SEQ. ID NO.8 are contained.

The recombinant vector provided in this application can be a recombinant vector prepared by linking the gene described above into any existing vector in the prior art in this field. In one specific example, the vector may be selected from at least one of a plasmid, a virus and a bacteriophage. Alternatively, the vector may be a AAV, such as an ssAAV.CMV.EGFP.WPRE.SV40pA vector.

A use of an intrabody described in any one of the above, a gene described above, a recombinant vector described above, an intrabody obtained by a preparation method described in any one of the above, or a recombinant expression vector described above is in any one of the following:
(1) in the preparation of a product for specifically recognizing and/or specifically binding to mutant HTT proteins and aggregates thereof;
(2) in the preparation of a product for degrading the mHTT proteins and aggregates thereof;
(3) in the preparation of a product for restoring lysosomal function; and
(4) in the preparation of a medicament for the treatment of a neurodegenerative disease.

In one specific example, the neurodegenerative disease is caused by the aggregate produced by the accumulation of mutant proteins, including, but not limited to HD, AD and PD.

Alternatively, the neurodegenerative disease may be HD, and an Intrabody fragment designed in this application can not only recognize a soluble mHTT, but also can recognize an insoluble mHTT aggregate, reducing mHTT and aggregates thereof at the protein level, which better alleviates HD pathology, and provides an effective treatment method for HD disease in patients with advanced disease, not just early disease.

One implementation of this application also provides a small molecule medicament including an intrabody described in any one of the above, an intrabody obtained by a preparation method described in any one of the above, a gene described in any one of the above, or a recombinant expression vector described above.

The antibody fragment selected in this application is very small, the whole Intrabody is only 153 bp, about 5.8 kDa, which is convenient for operation and easier to be made into small molecular medicaments and accepted by patients.

Hereinafter, implementations of this application will be described in detail with reference to embodiments. It is to be understood that these embodiments are for illustrative purposes only and are not intended to limit the scope of this application. In the following embodiments, experimental methods with no specific conditions are indicated, reference is preferably made to the guidelines given in this application, to experimental manuals or routine conditions in the art, to conditions suggested by the manufacturer, or to experimental methods known in the art.

In the following specific embodiments, there may be slight deviations within the weighing accuracy range in the measurement parameters related to raw material components, unless otherwise specified. Involving temperature and time parameters, acceptable deviations caused by the instrument test accuracy or operation accuracy are allowed.

### Embodiment 1

### 1. Construction of Recombinant Expression Plasmids:

antibody sequences of CDR1, CDR2 and CDR3 regions, named SM1, SM2 and SM3, were selected and fused with HA tags and LAMP1 signal sequences to construct recombinant expression plasmids scFv-mEM48-SM1, scFv-mEM48-SM2 and scFv-mEM48-SM3.

In CDR1 region (SM1):
a nucleotide sequence was,
SEQ.ID NO. 5
5′-AGACTGGAATGGATGGGCTACATAAGCTACGACGGTAGAAATAACT
ACAACCCAT CTCTCAAAAATCGAATCTCC-3′,;
and
an amino acid sequence was,
SEQ.ID NO. 1
RLEWMGYISYDGRNNYNPSLKNRIS,;
in CDR2 region (SM2):
a nucleotide sequence was,
SEQ.ID NO. 6
5′-AGAAATCGAATCTCCATCACTCGTGACACATCTAAACACCAGTTTT
TCCTGAAGT TGAATTCTG TGACTACTGAGGACACAGCTACATATT
AC-3′,;
and
an amino acid sequence was,
SEQ.ID NO. 2
RNRISITRDTSKHQFFLKLNSVTTEDTATYY,;
and
in CDR3 region (SM3):
a nucleotide sequence was,
SEQ.ID NO. 7
5′-GCTACATATTACTGTGCAGCTTACTACGGTAATACCGGGGATTACT
CTTCTATGGA CTACTGGGGCCAAGGC-3′,;
and
an amino acid sequence was,
SEQ.ID NO. 3
ATYYCAAYYGNTGDYSSMDYWGQG,.

Taking scFv-mEM48-SM3 as an example, the specific steps were as follows.

A sequence of the CDR3 region (SEQ. ID NO.7) of VH of a monoclonal antibody mEM48 specifically recognizing mHTTs, was selected. The sequence specifically recognized mHTT proteins and aggregates thereof, named SM3. Two HA tag proteins were added before the SM3 sequence to facilitate subsequent detection for Intrabody. A tag protein sequence was: 5′-TACCCTTACGACGTACCAGACTATGCTTACCCTTACGACGTACCAGACTATGCT-3′, SEQ. ID NO.11. After the SM3 sequence, a signal sequence of LAMP1 was added, so that Intrabody could be recognized in both directions. The signal sequence was: 5′-GGCTATCAGACCATC-3′, SEQ. ID NO.8.

Total nucleotide sequences of intrabody were:

scFv-mEM48-SM1:
SEQ.ID NO.12
5′-ATGGTTGACTACCCTTACGACGTACCAGACTATGCTTACCCTTACGACGTAC
CAG ACTATGCTA
GACTGGAATGGATGGGCTACATAAGCTACGACGGTAGAAATAACTACAACC
CATCTCTCAAAAATCGAATCTCCGGCTATCAGACCATCTGA-3′,;
scFv-mEM48-SM2:
SEQ.ID NO. 13
5′-ATGGTTGACTACCCTTACGACGTACCAGACTATGCTTACCCTTACGACGTAC
CAG ACTATGCTA
GAAATCGAATCTCCATCACTCGTGACACATCTAAACACCAGTTTTTCCTGA
AGTTGAATTCTGTGACTACTGAGGACACAGCTACATATTACGGCTATCAGACCATCTGA-3′,;
and
scFv-mEM48-SM3:
SEQ.ID NO. 14
5′-ATGGTTGACTACCCTTACGACGTACCAGACTATGCTTACCCTTACGACGTAC
CAG
ACTATGCTGCTACATATTACTGTGCAGCTTACTACGGTAATACCGGGGATTACTCTTCTAT
GGACTACTGGGGCCAAGGCGGCTATCAGACCATCTGA-3′,.

Amino acid sequences of intrabody were:

scFv-mEM48-SM1:
SEQ.ID NO. 15
MVDYPYDVPDYAYPYDVPDYARLEWMGYISYDGRNNYNPSLKNRISGYQ
TI*,;
scFv-mEM48-SM2:
SEQ .IDNO. 16
MVDYPYDVPDYAYPYDVPDYARNRISITRDTSKHQFFLKLNSVTTEDTA
TYYGYQTI*,;
and
scFv-mEM48-SM3:
SEQ.ID NO. 17
MVDYPYDVPDYAYPYDVPDYAATYYCAAYYGNTGDYSSMDYWGQGGYQT
I*,.

To ensure the correct expression of the sequences and ensure that there was no frame shift, no restriction enzyme cutting site was added in the target sequence. Because of very small linked fragments in this application, there was no need to pay special attention to its steric hindrance, in order not to increase the molecular weight of Intrabody. For this reason, this application did not insert a catenation sequence within the target sequence. Through the later experimental verification, it was found that various components of Intrabody also play a good role without adding the catenation sequence.

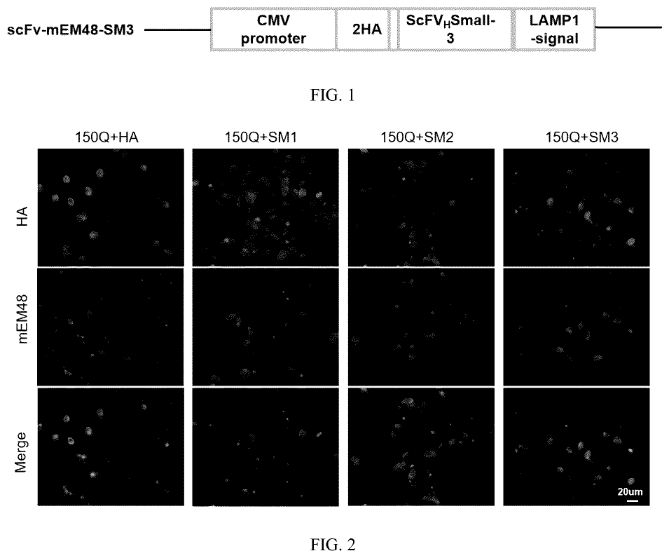

The fragment scFv-mEM48-SM3 was synthesized by using primers: 5′-CCGACCGGTGCCACCATGGTTGACTACC CT-3′ (SEQ. ID NO.9) and 5′-5′-CCGGAATTCGATTCAGATGGTCTGATA-3′ (SEQ. ID NO.10). Taking the synthetic fragment scFv-mEM48-SM3 as a template, scFv-mEM48-SM3 was amplified and EcoRI and AgeI restriction enzyme cutting sites were added. The amplified scFv-mEM48-SM3 was linked into a vector of SSAAV.CMV.EGFP.WPRE.SV40pA to replace an EGFP gene fragment, and an eukaryotic expression vector of ssAAV.CMV.2xHA-SM3.WPRE.SV40pA was constructed. The eukaryotic expression vector was transformed into Escherichia coli for expression and purification, and the plasmid scFv-mEM48-SM3 was successfully constructed. The schematic diagram of scFv-mEM48-SM3 was shown in FIG. 1.

### 2. Expression of Intrabody

The scFv-mEM48-SM3 was transformed into competent Escherichia coli, and the bacterial liquid was collected after the competent Escherichia coli with scFv-mEM48-SM3 was shaken at 37° C. at 220 rpm/min for 16 h; and after the bacterial liquid was centrifuged, the colony precipitate was collected and the expression of HA was detected, indicating that the intrabody was successfully expressed. At the same time, the scFv-mEM48-SM3 plasmid was injected into animals after being packaged with a virus. The expression of HA was detected, proving that the intrabody was successfully expressed.

### Embodiment 2

In the embodiment, a sequence of CDR region of VH of a monoclonal antibody mEM48 specifically recognizing mHTTs was screened at the level of 293T cells, and antibody sequences of CDR1, CDR2 and CDR3 regions, named SM1, SM2 and SM3, were selected and fused with HA tags and LAMP1 signal sequences to construct recombinant expression plasmids scFv-mEM48-SM1, scFv-mEM48-SM2 and scFv-mEM48-SM3, with the specific method referring to Embodiment 1.

293T cells were cultured, and when a growth density was 70-80%, an mHTT fragment (N171-150Q) containing 150Q at an N-terminal was co-transfected with scFv-mEM48-SM1, scFv-mEM48-SM2 and scFv-mEM48-SM3, and simultaneously, control plasmids were transfected as negative and positive controls.

That is, there were 5 groups:

(1) 293T+HA, (2) 293T+N171-150Q+HA, (3) 293T+N171-150Q+scFv-mEM48-SM1, (4) 293T+N171-150Q+scFv-mEM48-SM2, and (5) 293T+N171-150Q+scFv-mEM48-SM3.

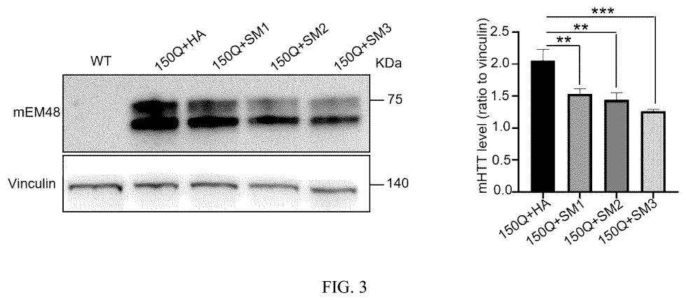

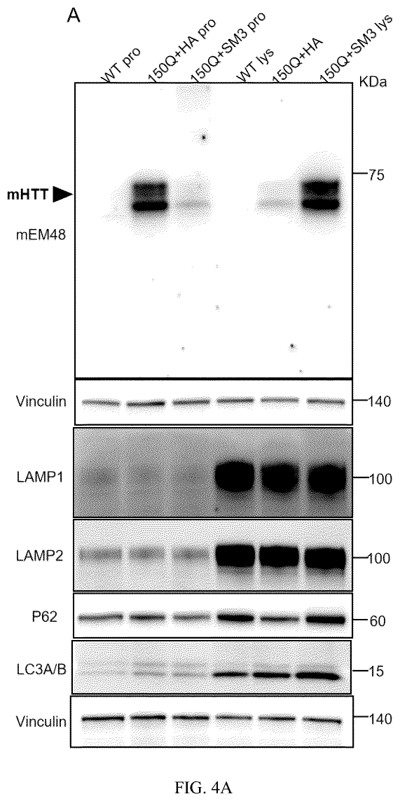

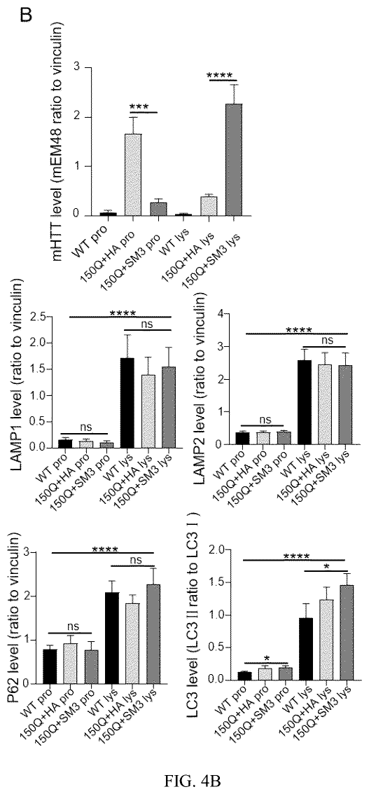

After 48 h of transfection, cells were collected for immunofluorescence and WB analysis to verify whether mHTTs were reduced, and a plasmid vector with the best effect was screened out. The results were shown in FIGS. 2-3, the levels of mHTTs transfected with scFv-mEM48-SM1, scFv-mEM48-SM2 and scFv-mEM48-SM3 were decreased to some extent; however, the level of mHTTs transfected with scFv-mEM48-SM3 was decreased more obviously. Therefore, the scFv-mEM48-SM3 plasmid vector was finally selected for subsequent experiments. Furthermore, cell lysosomes were extracted to explore whether mHTTs recognized and bound to by scFv-mEM48-SM3 (Intrabody) were carried into lysosomes. The results were shown as FIGS. 4A and 4B, indicating that more mHTTs were carried into lysosomes.

### Embodiment 3

In the embodiment, to eliminate the instability of transient transfection, the effect of scFv-mEM48-SM3 was verified in a stable cell line of 120Q-293T. 120Q-293T cells were cultured, and when a growth density was 70-80%, the scFv-mEM48-SM3 was transfected, and simultaneously, control plasmids were transfected as negative and positive controls.

That is, there were 3 groups:

(1) 23Q-293T+HA, (2) 120Q-293T+HA, and (3) 120Q-293T+scFv-mEM48-SM3.

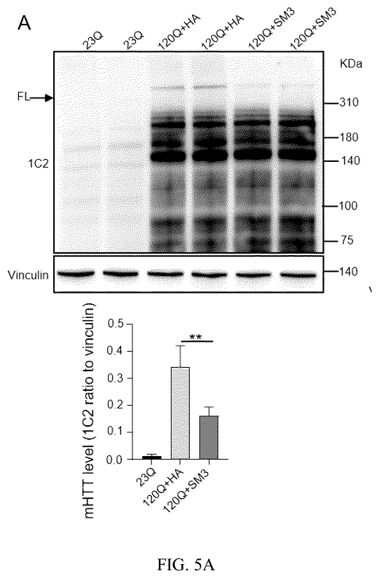

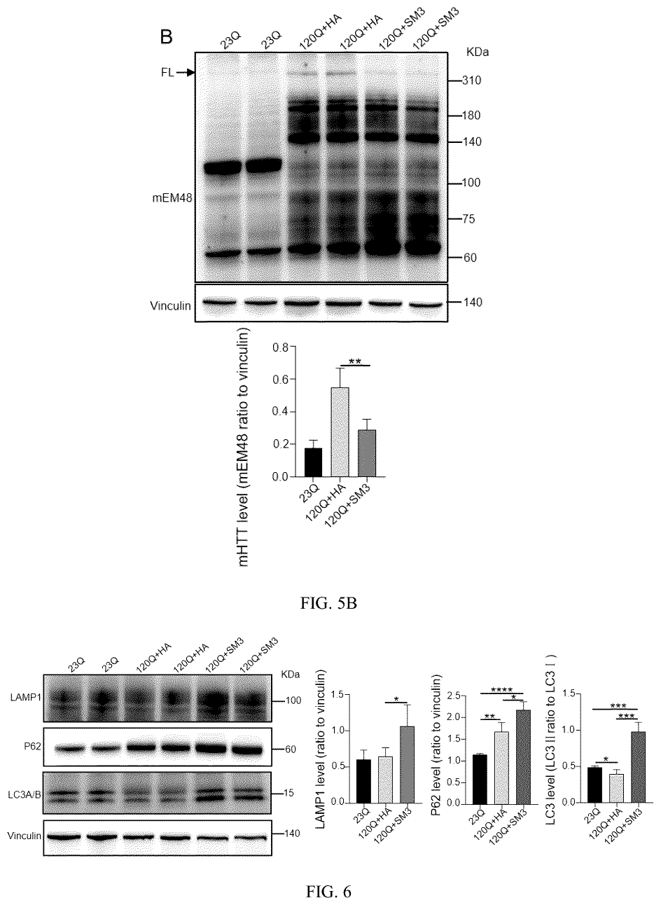

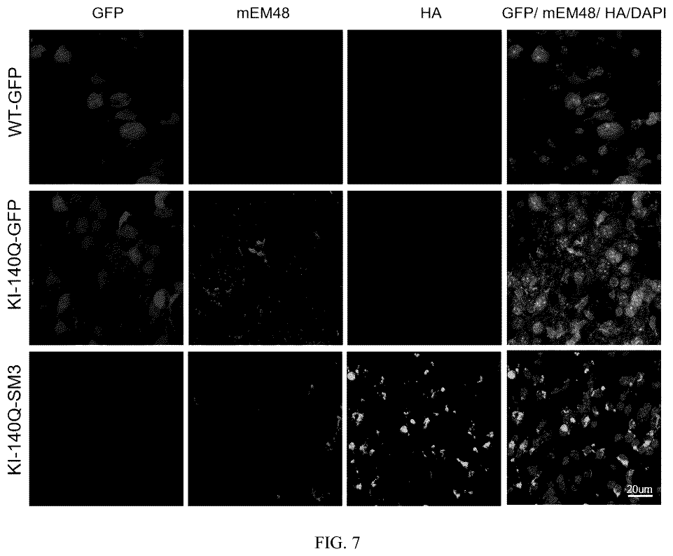

After 48 h of transfection, cells were collected for WB analysis to verify whether mHTTs were reduced. The results were shown as FIGS. 5A and 5B, finding that mHTTs were significantly reduced. Then the lysosome-associated proteins were incubated to verify whether lysosomes were activated. The results were shown in FIG. 6, indicating that the lysosomes were successfully activated after transfection with scFv-mEM48-SM3.

### Embodiment 4

In the embodiment, the effect of scFv-mEM48-SM3 was verified at the mouse level by brain injection.

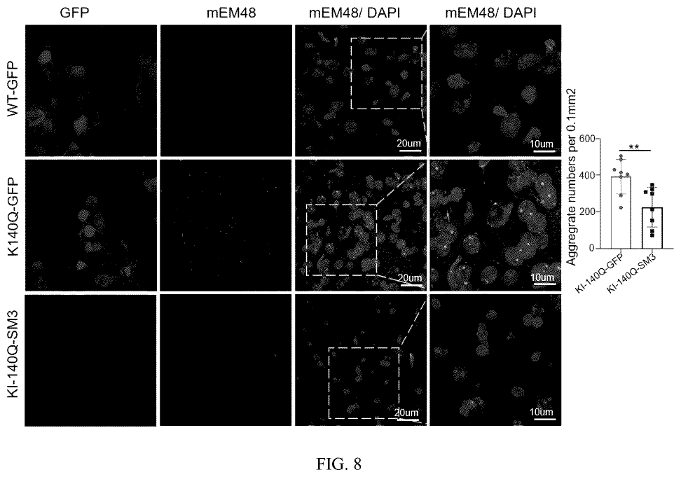

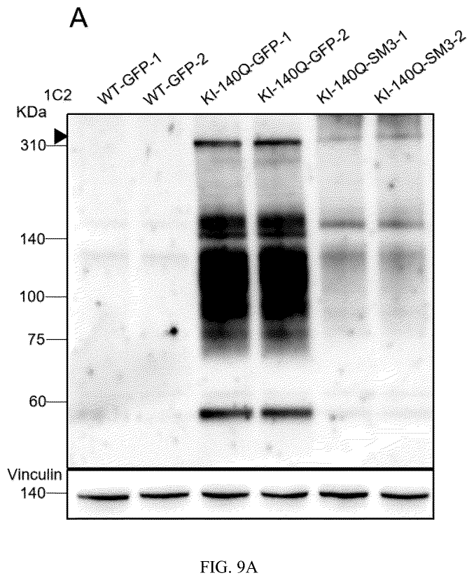

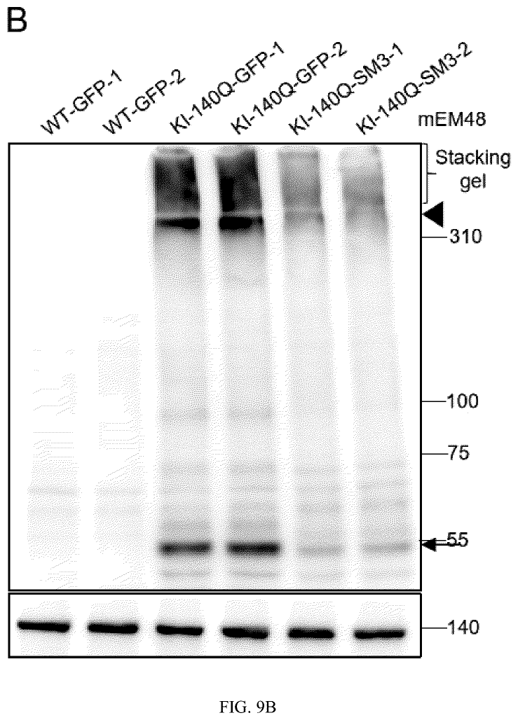

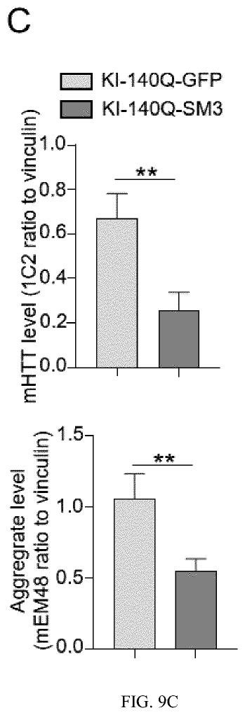

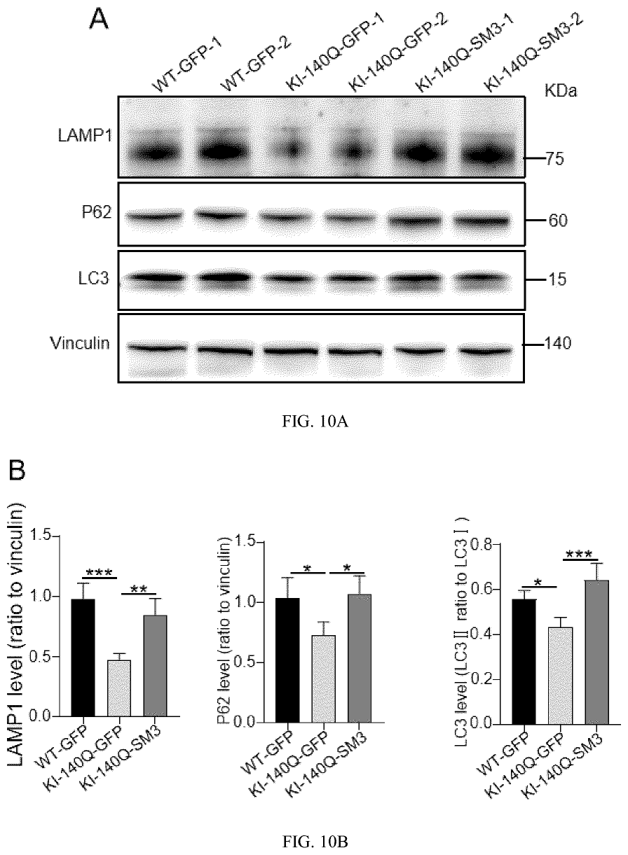

An scFv-mEM48-SM3 plasmid was packaged with an AAV virus, and HD-KI-140Q (HD gene knock-in mouse model) mice aged 6 months were selected for stereotactic brain injection. An AAV virus vector packaged with scFv-mEM48-SM3 was injected into the striatum parenchyma of mice, and simultaneously, an AAV-GFP virus was injected as a control virus. Mice were euthanized one month after injection. Firstly, the immunofluorescence analysis was carried out to detect whether the virus was effectively expressed. The results as shown in FIG. 7 showed that the virus was effectively expressed. Then, the immunofluorescence and WB analysis were carried out to detect whether mHTT proteins and aggregates thereof were reduced. The results of immunofluorescence as shown in FIG. 8 and western blot analysis as shown in FIGS. 9A-9C showed that the mHTT proteins and aggregates thereof were obviously reduced. Moreover, the lysosome-associated proteins were incubated to verify whether the lysosomes were activated. The results of WB were shown in FIG. 10, indicating that the lysosomes were successfully activated.

### Embodiment 5

In the embodiment, the effect of scFv-mEM48-SM3 was verified at the mouse level by orbital intravenous injection.

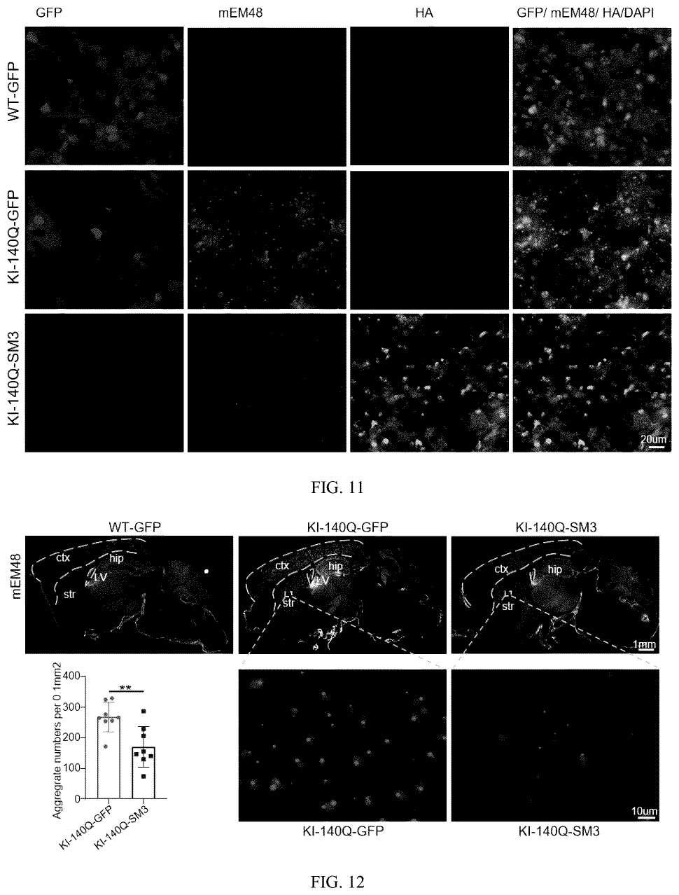

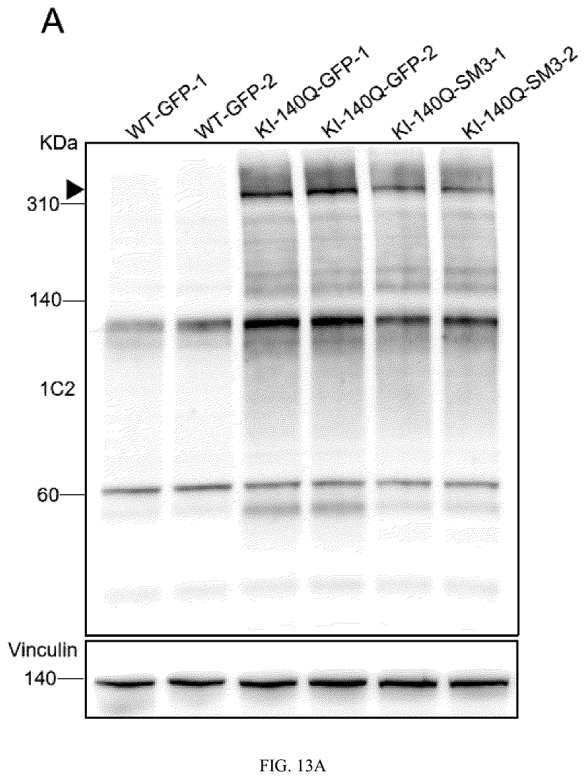

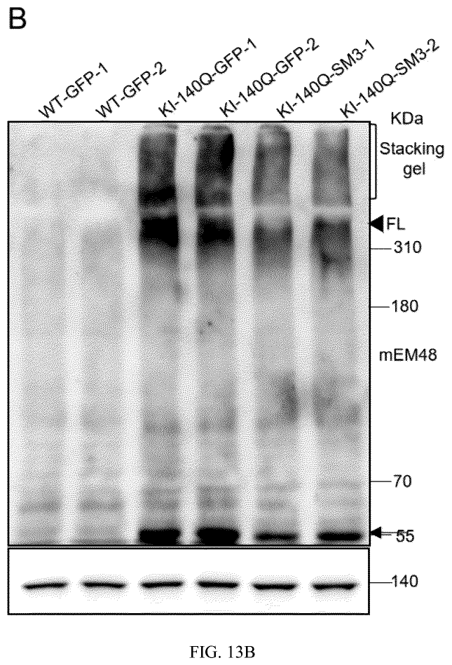

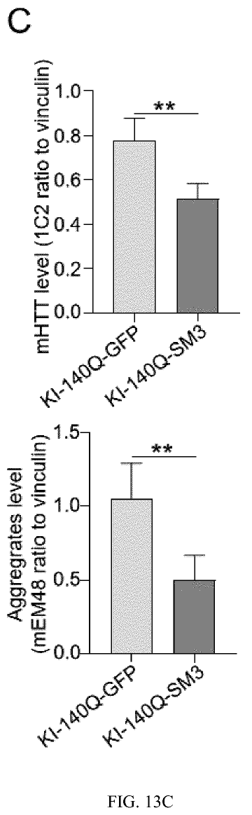

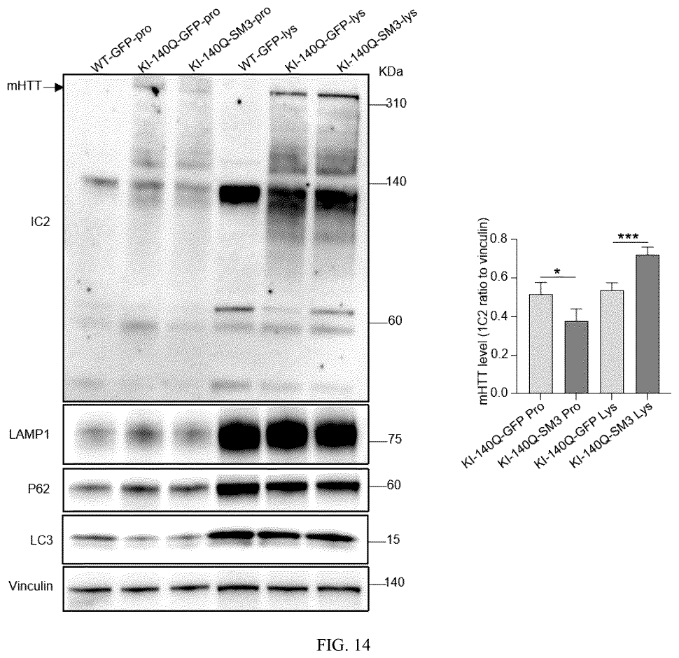

An scFv-mEM48-SM3 plasmid was packaged with an AAV-PHP.eB serotype virus, and HD-KI-140Q mice aged 6 months were selected for orbital injection. An AAV-GFP-PHP.eB virus vector packaged with scFv-mEM48-SM3 was delivered to the whole brain via orbital vein, and mice were euthanized one month after injection. Firstly, the immunofluorescence analysis was carried out to detect whether the virus was effectively expressed. The results showed that the virus was effectively expressed in the brain as shown in FIG. 11. Then, the immunofluorescence and WB analysis were carried out to detect whether mHTT proteins and aggregates thereof were reduced. The results were shown in FIGS. 12-13, indicating that ScFv-mEM48-SM3 could also effectively reduce the level of mHTTs by intravenous injection. Furthermore, tissue lysosomes were extracted to explore whether mHTTs recognized and bound to by scFv-mEM48-SM3 were brought into lysosomes. As shown in FIG. 14, the results showed that Intrabody had a good bidirectional recognition function.

### Embodiment 6

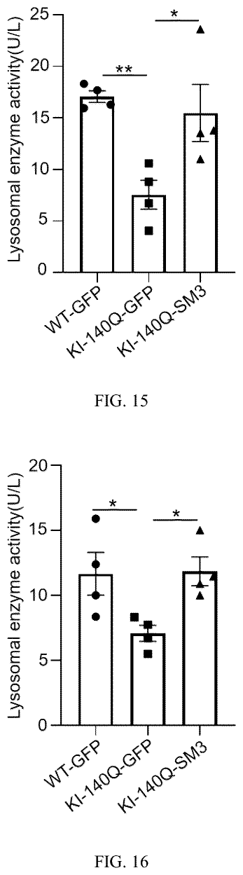

In the embodiment, whether mHTTs damage lysosomes or affect the enzyme activity of lysosomes after being brought into lysosomes by scFv-mEM48-SM3 was explored at the mouse level. Therefore, tissue samples of brain injection and intravenous injection were selected for ELISA to determine the total acid enzyme activity in lysosomes. The results showed that the enzyme activities of lysosomes in the striatums of HD-KI-140Q mice were obviously restored by brain injection (seen in FIG. 15) and intravenous injection (seen in FIG. 16) after treatment with scFv-mEM48-SM3. The results showed that the Intrabody designed in this application could not only directionally degrade toxic proteins, but also restore lysosomal function.

The technical features of the aforementioned embodiments can be combined as desired, and not all possible combinations of the technical features in the aforementioned embodiments are described for the sake of brevity. However, as long as the combination of these technical features is not contradictory, it should be considered within the scope outlined in this specification.

The above embodiments represent only a few implementations of this application and are described in a more specific and detailed manner. However, they should not be interpreted as limiting the scope of the disclosed patents. It should be noted that individuals skilled in the art can make several modifications and improvements without deviating from the concept of this application, which fall within the protective scope of this application. Therefore, the scope of patent protection in this application is determined by the appended claims, and the description and drawings are provided to explain the contents of the claims.

1. An intracellular antibody (intrabody), comprising a fragment of a variable region of heavy chain (VH) of a monoclonal antibody recognizing a mutant huntingtin (mHTT) and a signal sequence of a lysosome-associated membrane protein I (LAMP1), an amino acid sequence of the intrabody being shown in any one of SEQ. ID NO.15-17; and
a preparation method for an intrabody, comprising the steps of:
constructing a recombinant expression vector containing a nucleotide sequence shown in any one of SEQ. ID NO.12-14; and
transforming the recombinant expression vector into a recipient bacterium or recipient cell for culture and expression to obtain an intrabody.

2. The intrabody according to claim 1, wherein a construction method for the recombinant expression vector comprises the steps of:
synthesizing a target fragment, the target fragment being the nucleotide sequence shown in any one of SEQ. ID NO.12-14;
taking the target fragment as a template, taking nucleotide sequences shown in SEQ. ID NO.18-19, SEQ. ID NO. 20-21 and SEQ. ID NO. 9-10 as primers of the target fragment shown in SEQ. ID NO.12-14 to amplify the target fragment, and adding EcoRI and AgeI restriction enzyme cutting sites; and
linking an amplified target fragment into an expression vector to obtain a recombinant expression vector.

3. A gene encoding an intrabody according to claim 1.

4. A recombinant expression vector containing a gene according to claim 3.

5. The recombinant expression vector according to claim 4, wherein the recombinant expression vector is selected from at least one of a plasmid and a virus.

6. The recombinant expression vector according to claim 5, wherein the virus comprises a bacteriophage.

7. A use of an intrabody according to claim 1 in any one of the following:
(1) in the preparation of a product for specifically recognizing and/or specifically binding to mHTT proteins and aggregates thereof;
(2) in the preparation of a product for degrading the mHTT proteins and aggregates thereof;
(3) in the preparation of a product for restoring lysosomal function; and
(4) in the preparation of a medicament for the treatment of Huntington's disease (HD).

8. A use of a gene according to claim 3 in any one of the following:
(1) in the preparation of a product for specifically recognizing and/or specifically binding to mHTT proteins and aggregates thereof;
(2) in the preparation of a product for degrading the mHTT proteins and aggregates thereof;
(3) in the preparation of a product for restoring lysosomal function; and
(4) in the preparation of a medicament for the treatment of HD.

9. A use of a recombinant expression vector according to claim 4 in any one of the following:
(1) in the preparation of a product for specifically recognizing and/or specifically binding to mHTT proteins and aggregates thereof;
(2) in the preparation of a product for degrading the mHTT proteins and aggregates thereof;
(3) in the preparation of a product for restoring lysosomal function; and
(4) in the preparation of a medicament for the treatment of HD.

10. A small molecule medicament, comprising an intrabody obtained by a preparation method according to claim 1.


SEQUENCE LISTING (21 sequences)

SEQ ID NO: 1 - protein, 25 aa, synthetic construct
RLEWMGYISYDGRNNYNPSLKNRIS

SEQ ID NO: 2 - protein, 31 aa, synthetic construct
RNRISITRDTSKHQFFLKLNSVTTEDTATYY

SEQ ID NO: 3 - protein, 24 aa, synthetic construct
ATYYCAAYYGNTGDYSSMDYWGQG

SEQ ID NO: 4 - protein, 5 aa, synthetic construct
GYQTI

SEQ ID NO: 5 - DNA, 75 bp, synthetic construct
  source 1..75: mol_type = other DNA
agactggaatggatgggctacataagctacgacggtagaaataactacaacccatctctcaaaaatcgaatctcc

SEQ ID NO: 6 - DNA, 93 bp, synthetic construct
  source 1..93: mol_type = other DNA
agaaatcgaatctccatcactcgtgacacatctaaacaccagtttttcctgaagttgaattctgtgactactgaggacacagctacatattac

SEQ ID NO: 7 - DNA, 72 bp, synthetic construct
  source 1..72: mol_type = other DNA
gctacatattactgtgcagcttactacggtaataccggggattactcttctatggactactggggccaaggc

SEQ ID NO: 8 - DNA, 15 bp, synthetic construct
  source 1..15: mol_type = other DNA
ggctatcagaccatc

SEQ ID NO: 9 - DNA, 30 bp, synthetic construct
  source 1..30: mol_type = other DNA
ccgaccggtgccaccatggttgactaccct

SEQ ID NO: 10 - DNA, 27 bp, synthetic construct
  source 1..27: mol_type = other DNA
ccggaattcgattcagatggtctgata

SEQ ID NO: 11 - DNA, 54 bp, synthetic construct
  source 1..54: mol_type = other DNA
tacccttacgacgtaccagactatgcttacccttacgacgtaccagactatgct

SEQ ID NO: 12 - DNA, 156 bp, synthetic construct
  source 1..156: mol_type = other DNA
atggttgactacccttacgacgtaccagactatgcttacccttacgacgtaccagactatgctagactggaatggatgggctacataagctacgacggtagaaataactacaacccatctctcaaaaatcgaatctccggctatcagaccatctga

SEQ ID NO: 13 - DNA, 174 bp, synthetic construct
  source 1..174: mol_type = other DNA
atggttgactacccttacgacgtaccagactatgcttacccttacgacgtaccagactatgctagaaatcgaatctccatcactcgtgacacatctaaacaccagtttttcctgaagttgaattctgtgactactgaggacacagctacatattacggctatcagaccatctga

SEQ ID NO: 14 - DNA, 153 bp, synthetic construct
  source 1..153: mol_type = other DNA
atggttgactacccttacgacgtaccagactatgcttacccttacgacgtaccagactatgctgctacatattactgtgcagcttactacggtaataccggggattactcttctatggactactggggccaaggcggctatcagaccatctga

SEQ ID NO: 15 - protein, 51 aa, synthetic construct
MVDYPYDVPDYAYPYDVPDYARLEWMGYISYDGRNNYNPSLKNRISGYQTI

SEQ ID NO: 16 - protein, 57 aa, synthetic construct
MVDYPYDVPDYAYPYDVPDYARNRISITRDTSKHQFFLKLNSVTTEDTATYYGYQTI

SEQ ID NO: 17 - protein, 50 aa, synthetic construct
MVDYPYDVPDYAYPYDVPDYAATYYCAAYYGNTGDYSSMDYWGQGGYQTI

SEQ ID NO: 18 - DNA, 30 bp, synthetic construct
  source 1..30: mol_type = other DNA
ccgaccggtgccaccagactggaatggatg

SEQ ID NO: 19 - DNA, 28 bp, synthetic construct
  source 1..28: mol_type = other DNA
ccgcaattcgatggagattcgatttttg

SEQ ID NO: 20 - DNA, 30 bp, synthetic construct
  source 1..30: mol_type = other DNA
ccgaccggtgccaccagaaatcgaatctcc

SEQ ID NO: 21 - DNA, 27 bp, synthetic construct
  source 1..27: mol_type = other DNA
ccggaattcgatgtaatatgtagctgt